(19) United States
(12) Patent Application Publication
(10) Pub. No.: US 2018/0125774 A1
(43) Pub. Date: May 10, 2018

(54) Method and Composition for Visible Light Dryable Nail Coatings

(71) Applicant: Lijuan Zhen, City of Industry, CA (US)

(72) Inventor: Lijuan Zhen, City of Industry, CA (US)

(21) Appl. No.: 15/486,314

(22) Filed: Apr. 13, 2017

Related U.S. Application Data
(60) Provisional application No. 62/418,751, filed on Nov. 7, 2016.

(51) Int. Cl. A61K 8/87; A61K 8/58; A61K 8/41; A61Q 3/02; B08B 3/08

(52) U.S. Cl. CPC A61K 8/87; A61K 8/58; A61K 2800/81; A61Q 3/02; B08B 3/08; A61K 8/411

(57) ABSTRACT

A composition for visible light dryable nail coating includes a base acrylate copolymer; a photo-initiator; and a photo-initiator monomer; wherein the photo-initiator is able to initiate natural light polymerization of chemically unsaturated monomers and prepolymers. A method for applying a visible light dryable nail coating includes steps of: applying a visible light dryable nail coating on a nail surface; curing the visible light dryable nail coating by a light source; and removing the visible light dryable nail coating from the nail surface in a solvent.

### CROSS REFERENCE OF RELATED APPLICATION

This is a non-provisional application that claims the benefit of priority under 35U.S.C.§ 120 to a provisional application, application No. 62/418,751, filed Nov. 7, 2016. The afore-mentioned provisional application is hereby incorporated by reference in its entirety.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention related to a nail coating, and more particularly to a visible light dryable nail coating which not only provide a smooth, hard, and glossy coating, but also can be curved by exposure to natural light.

### Description of Related Arts

Nowadays, various nail coatings are prepared and marketed in the current market, wherein the common nails coatings are classified into two categories: nail polishes, such as lacquers, varnish or enamels and artificial nails which are known as gels or acrylics. The composition of the nail polishes is various solid components which are dissolved in aromatic solvent. Upon application and drying, the nail polishes can be dried out by blowing or circulating hot or cold air on the nail coating for a specified period of time. However, the above mentioned nail polishes are easily to be scratched and are easily to chip out within few days.

U.S. Pat. No. 5,456, 905 and U.S. Pat. No. 5,435, 994 to Valenty (1995) disclose an improved nail coating which provides a smooth, glossy, and hard nail coating, wherein the improved nail coating comprises a base resin, a photo-reactive monomer, a photo-initiator, and an inhibitor, which are typically 100% solids and does not require aromatic solvents. Upon application and drying, the improved nail coating is long lasting, hardy, and highly durable nail coating. In addition, in the formation of the improved nail coating, the nail coating is formed by exposure to UV radiation. However, it is harmful for the human's skin being frequently exposure to UV radiation, but if the improved nail coating is not exposed under UV radiation, only the surface of the improved nail coating is curved, and the underneath of the improved nail coatings do not be cured. Therefore, the improved nail coating wearers still need to use their hands carefully.

Furthermore, since the above mentioned inherent properties, the improved nail coatings are not easily to be removed. Upon the removal of the improved nail coating, the improved nail coating requires to be soaked in non-reactive solvent for at least 30 to 90 minutes, after that a wooden or metal probe is required to heavily abrade or scrap the improved nail coatings. During the removal of the improved nail coating, the human's nails are easily to be damaged by the removal process.

### SUMMARY OF THE PRESENT INVENTION

The invention is advantageous in that it provides a visible light dryable nail coating which is able to be cured by exposure to natural light, and can be peel off to remove, or can be wiped off or soak off from a nail surface by a solvent.

Another advantage of the invention is to provide a visible light dryable nail coating which comprises a base acrylates copolymer, a photo-initiator, and a photo-initiator monomer, wherein the photo-initiator can be component (A): visible light photo-initiator A, and (B): visible light photo initiator B. The wherein said photo-initiator monomer is selected from the group consisting of at least an acrylate monomer, a methacrylate monomer, a triethylene glycol dimethacrylate, a hydroxypropyl methacrylate, an isobornyl methacrylate, an isobornyl acrylate, a hydroxyethyl methacrylate (HEMA), hema maleate, and a PEG-4 dimethacrylate, ethyl methacrylate, and dicyclopentadienylacrylate.

Another advantage of the invention is to provide a visible light dryable nail coating, wherein the visible light photo-initiator A is, but not limited to, Bis(η5-2,4-cylcopentadian-1-yl)-bis(2,6-di-fluoro-3-(1H-pyrrol-1-yl)phenyl)titanium which is able to initiate UV and/or visible light polymerization of chemically unsaturated monomers and prepolymers with an absorption between 350 to 450 nm.

Another advantage of the invention is to provide a visible light dryable nail coating, wherein the visible light photo-initiator B, but not limited to, 4,4′-Bis (diethyl amino) benzophenone, and acts both as a photosensitizer and an amine synergist with a strong absorption between 400 to 450 nm.

Another advantage of the invention is provided to a visible light dryable nail coating, wherein the base acrylates copolymer is a film forming agent which can be embodied as urethane acrylate, Aliphatic urethane diacrylate, Aliphatic urethane acrylate, modified solvent based urethane acrylate, Aliphatic urethane Hexaacrylate, silicone modified urethane acrylate, and the like.

Another advantage of the invention is to provide a visible light dryable nail coating, wherein the visible light dryable nail coating is easily removed in just a single piece or can be wiped off or soak off from a nail surface by a solvent.

Additional advantages and features of the invention will become apparent from the description which follows, and may be realized by means of the instrumentalities and combinations particular point out in the appended claims.

According to the present invention, the foregoing and other objects and advantages are attained by a composition of a visible light dryable nail coating, comprising:

a base acrylate copolymer;

a photo-initiator; and

a photo-initiator monomer; wherein

the photo-initiator can be, but not limit to: either Bis(η5-2,4-cylcopentadian-1-yl)-bis(2,6-di-fluoro-3-(1H-pyrrol-1-yl)phenyl)titanium or 4,4′-Bis (diethylamino) benzophenone. The wherein said photo-initiator monomer is selected from the group consisting of at least an acrylate monomer, a methacrylate monomer, a triethylene glycol dimethacrylate, a hydroxypropyl methacrylate, an isobornyl methacrylate, an isobornyl acrylate, a hydroxyethyl methacrylate (HEMA), hema maleate, and a PEG-4 dimethacrylate, ethyl methacrylate, and dicyclopentadienylacrylate.

In accordance with another aspect of the invention, the present invention comprises a method for applying a visible light dryable nail coating, comprising steps of:

(1) applying a visible light dryable nail coating on a nail surface;

(2) curving the visible light dryable nail coating by a light source; and

(3) removing the visible light dryable nail coating from the nail surface in a solvent.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a block diagram of a method for applying a visible light dryable nail coating according to a second preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

The present invention discloses a composition for a visible light dryable nail coating according to a first preferred embodiment of the present invention, wherein the visible light dryable nail coating can be curved by any electromagnetic waves or particle beams. The composition of the visible light dryable nail coating comprises a base acrylates copolymer, a photo-initiator, and a photo-initiator monomer. The method of mixing and preparation of the resulting composition is obvious in the prior art.

The base acrylates copolymer is a film forming agent which can be embodied as urethane acrylate, Aliphatic urethane diacrylate, Aliphatic urethane acrylate, modified solvent based urethane acrylate, Aliphatic urethane Hexaacrylate, silicone modified urethane acrylate, and the like. The photo-initiator monomer is selected from the group consisting of one or more acrylate monomer, methacrylate monomer, triethylene glycol dimethacrylate, hydroxypropyl methacrylate, isobornyl methacrylate, isobornyl acrylate, hydroxyethyl methacrylate (HEMA), hema maleate, and PEG-4 dimethacrylate, ethyl methacrylate, and dicyclopentadienylacrylate.

Any one of several photo-initiators is used in the present invention, wherein the photo-initiators can be either component (A): visible light photo-initiator A or component (B): visible light photo-initiator B. Visible light photo-initiator A is, but not limited to, Bis (η5-2,4-cylcopentadian-1-yl)-bis (2,6-di-fluoro-3-(1H-pyrrol-1-yl) phenyl) titanium. Visible light photo-initiator A is a highly efficient orange colored photo-initiator with good thermal stability which is able to initiate UV and/or visible light polymerization of chemically unsaturated monomers and prepolymers, and belongs to the class of organometallic photo-initiator and can be used for both free-radical and cationic systems. More specifically, visible light photo-initiator A is particularly suitable for the curing of photopolymers for imaging or information storage applications where extremely high photosensitivity is required.

The following table 1 lists the specification of the visible light photo-initiator A of the first preferred embodiment of the visible light dryable nail coating according to this present invention.

TABLE 1

| | | Assay | | |
|---|---|---|---|---|
| Chemical Name | Appearance | (HPLC) | Melting point | Volatiles |
| Visible light | Yellow to | 99% min. | 180° C. min. | 0.5% max. |
| photo-initiator A | orange | | | |
| | powder | | | |

The following table 2 lists the solubility of the visible light photo-initiator A of the first preferred embodiment of the visible light dryable nail coating according to this present invention.

TABLE 2

| Solubility | g in 100 ml solvent @25° C. |
|---|---|
| Acetone | 30 |
| Toluene | 10 |
| MEK | 30 |
| HDDA | 10 |
| Water | <0.5 |
| Butyl Acetate | 2 |

It is worth to mention that visible light photo-initiator A is sensitive to visible light and any exposure to sunlight must be avoided. And, the visible light photo-initiator A must be stored in low temperature, which is below 25° C., and dry environment. When visible light photo-initiator A is dissolved in a solvent or a formulation, it is extremely sensitive to daylight and light from standard fluorescence bulbs, so any open manipulation of visible light photo-initiator A must be carried out either in the dark or under light provided by a suitable red light sources. Therefore, opened drums are required to close after use to protect the product against light.

Accordingly, visible light photo-initiator B is, but not limited to, 4,4′-Bis (diethylamino) benzophenone. Visible light photo-initiator B is both an aromatic ketone and a tertiary amine, and acts both as a photosensitizer and an amine synergist. It is worth to mention that visible light photo-initiator B has a strong absorption for a light source having a wavelength between 350 to 450 nm, which is the most suitable for pigmented and black UV coating, and is a photo-grade EMK which bears bright yellow color and is more resistant to oxidation by exposure in air than most commercially available EMK.

The following table 3 lists the chemical information of the visible light photo-initiator B of the first preferred embodiment of the visible light dryable nail coating according to this present invention.

TABLE 3

| | |
|---|---|
| EINECS No. | 202-025-4 |
| Molecular formula | C₂₁H₂₈NO₂ |
| Molecular weight | 324.4 |
| Physical Data | |
| Odor | Very faint |
| .ε @363 nm | 3.8 × 104 mL mol⁻¹ cm⁻¹ |

The following table 4 lists the specification of the visible light photo-initiator B of the first preferred embodiment of the visible light dryable nail coating according to this present invention.

TABLE 4

| | |
|---|---|
| Appearance | Off-white with blueish tint |
| Assay (HPLC) | 98% min. |
| Melting point | 92° C. min. |
| Solubility (3 g/10 mL Acetone) | Clear solution |

The following table 5 lists the solubility of the visible light photo-initiator B of the first preferred embodiment of the visible light dryable nail coating according to this present invention.

TABLE 5
Solubility (% w/w @20° C.)

| | |
|---|---|
| Water | <0.01 |
| Acetone | 30 |
| Methanol | 7.0 |
| Toluene | 25 |
| Ethyl acetate | 24 |
| Butyl Acetate | 30 |

Accordingly, the usage rates of visible light photo-initiator B vary according to the composition of the system, source of light, line speed, and film thickness, and usually lie between 0.25% to 3% w/w together with photo-activators at 0.2-1.5% w/w. visible light photo-initiator B is especially suitable for applications where curing extent is critical but color of appearance is not essential such as for photoresist dry films. In addition, the use of visible light photo-initiator B in pigmented system, such as printing inks, results the increase of curing speed and surface drying.

According to the preferred embodiment of the present invention, the visible light dryable nail coating of the present invention can be curved by exposure to electromagnetic waves, which has wavelengths between 400-450 nm, wherein the electromagnetic waves can be natural light, including any kinds of light source in our daily life. Therefore, the visible light dryable nail coatings don't need to be dried out by air or any thermal dry device.

The photo-initiator monomer is adapted to provide the polymerized composition increased adhesion, wherein the photo-initiator monomer can be any type of monomer which is suitable for use within a composition for forming a nail coating. In addition, the photo-initiator monomer can be, but not limited to, one or more of acrylate monomer, methacrylate monomer, triethylene glycol dimethacrylate, hydroxypropyl methacrylate, isobornyl methacrylate, isobornyl acrylate, hydroxyethyl methacrylate (HEMA), hema maleate, PEG-4 dimethacrylate, ethyl methacrylate, or dicyclopentadienylacrylate.

It is worth to mention that the visible light dryable nail coating is easily removed by wiping off, even where the coating comes into contact with the cuticle of the finger nails, and the present invention still achieving the hardness and gloss similar to present nitrocellulose based nail lacquers. The visible light dryable nail coating of the present invention can be easily peeled off from the fingernails or can be wipe off or soak off from a nail surface by a solvent.

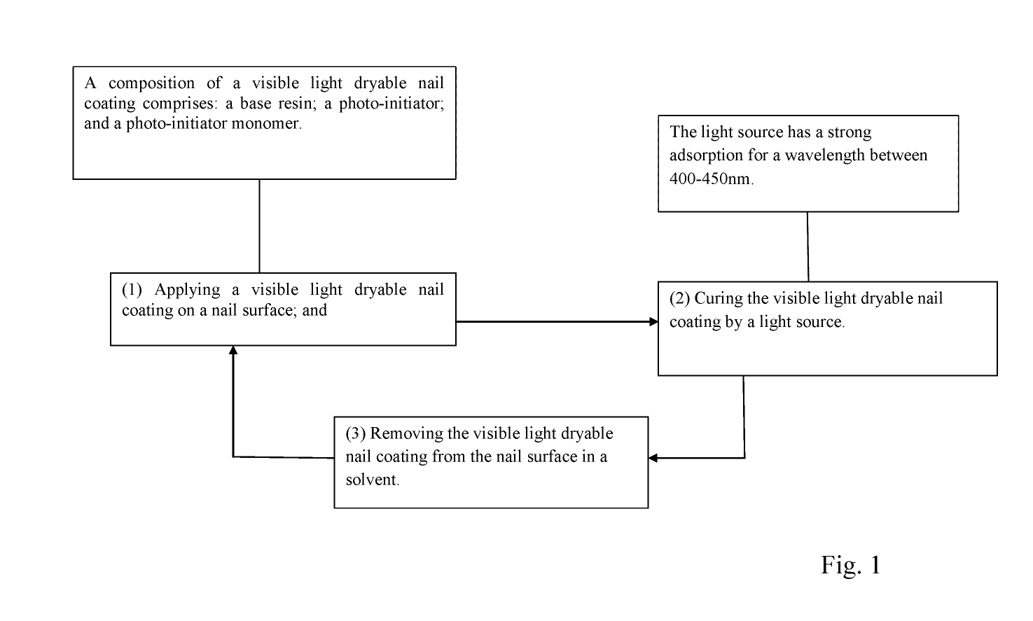

Referring to FIG. 1, a method for applying a visible light dryable nail coating according to a second preferred embodiment of the present invention is illustrated, wherein the method comprises the following step:

(1) applying a visible light dryable nail coating on a nail surface; and

(2) curing the visible light dryable nail coating by a light source.

In the step (1), the composition of the visible light dryable nail coating comprises a base acrylates copolymer, a photo-initiator, and a photo-initiator monomer, wherein the bas acrylates copolymer is curved by UV, visible light, EB, or heat. The method of mixing and preparation of the resulting composition is obvious in the prior art.

The base acrylates copolymer is a film forming agent which can be embodied as urethane acrylate, Aliphatic urethane diacrylate, Aliphatic urethane acrylate, modified solvent based urethane acrylate, Aliphatic urethane Hexaacrylate, silicone modified urethane acrylate, and the like. The photo-initiator monomer is selected from the group consisting of at least an acrylate monomer, a methacrylate monomer, a triethylene glycol dimethacrylate, a hydroxypropyl methacrylate, an isobornyl methacrylate, an isobornyl acrylate, a hydroxyethyl methacrylate (HEMA), a hema maleate, a PEG-4 dimethacrylate, an ethyl methacrylate, and a dicyclopentadienylacrylate.

Any one of several photo-initiators is used on the present invention, wherein the photo-initiators can be either component (A): visible light photo-initiator A or component (B): visible light photo-initiator B. And, the specification, physical data, solubility, appearance, and other chemical and physical properties are the same as mentioned above the first preferred embodiment.

In the step (1), the photo-initiator monomer is adapted to provide the polymerized composition increased adhesion, wherein the photo-initiator monomer can be any type of monomer which is suitable for use within a composition for forming a nail coating. In addition, the photo-initiator can be, but not limited to, one or more of triethylene glycol dimethacrylate, hydroxypropyl methacrylate, isobornyl methacrylate, isobornyl acrylate, hydroxyethyl methacrylate (HEMA), hema maleate, PEG-4 dimethacrylate, ethyl methacrylate, or dicyclopentadienylacrylate. The photo-initiator monomer is selected from the group consisting of one or more acrylate monomer, methacrylate monomer, triethylene glycol dimethacrylate, hydroxypropyl methacrylate, isobornyl methacrylate, isobornyl acrylate, hydroxyethyl methacrylate (HEMA), hema maleate, PEG-4 dimethacrylate, ethyl methacrylate, and dicyclopentadienylacrylate.

In the step (2), the visible light dryable nail coating can be curved by any electromagnetic waves or particle beams which have wavelengths between 350-450 nm, wherein the electromagnetic waves can be natural light, including any kinds of light source in our daily life. Therefore, the visible light dryable nail coatings are not need to be dried out by air or any thermal dry device.

The method further comprises a step (3): removing the visible light dryable nail coating from the nail surface in a solvent.

In the step (3), the visible light dryable nail coating is easily removed by peeling in just a single piece, even where the coating comes into contact with the cuticle of the finger nail, and the present invention still achieving the hardness and gloss similar to present nitrocellulose based nail lacquers. The visible light dryable nail coating of the present invention can be easily removed from the fingernails by soaking the visible light dryable nail coating into solvent commonly used for nail polish removal.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A composition for visible light dryable nail coating, comprising:
a base acrylate copolymer;
a photo-initiator; and
a photo-initiator monomer; wherein said photo-initiator is able to initiate natural light polymerization of chemically unsaturated monomers and prepolymers.

2. The composition, as recited in claim 1, wherein said photo-initiator is, but not limited to, Bis (η5-2,4-cylcopentadian-1-yl)-bis (2,6-di-fluoro-3-(1H-pyrrol-1-yl) phenyl) titanium.

3. The composition, as recited in claim 1, wherein said photo-initiator is, but not limited to, 4,4′-Bis (diethylamino) benzophenone.

4. The composition, as recited in claim 1, wherein said photo-initiator has a strong absorption between 400 to 450 nm.

5. The composition, as recited in claim 1, wherein said visible light dryable nail coating can be cured by exposure to natural light.

6. The composition, as recited in claim 2, wherein said base acrylate copolymer which is the film forming agent.

7. The composition, as recited in claim 6, wherein said film forming agent is selected from the group consisting of urethane acrylate, Aliphatic urethane diacrylate, Aliphatic urethane acrylate, modified solvent based urethane acrylate, Aliphatic urethane Hexaacrylate, and silicone modified urethane acrylate.

8. The composition, as recited in claim 3, wherein said base acrylate copolymer is cured by visible light, EB or heat.

9. The composition, as recited in claim 7, wherein said photo-initiator monomer is selected from the group consisting of one or more acrylate monomer, methacrylate monomer, triethylene glycol dimethacrylate, hydroxypropyl methacrylate, isobornyl methacrylate, isobornyl acrylate, hydroxyethyl methacrylate (HEMA), hema maleate, PEG-4 dimethacrylate, ethyl methacrylate, and dicyclopentadienylacrylate.

10. The composition, as recited in claim 8, wherein said photo-initiator monomer is selected from the group consisting of one or more acrylate monomer, methacrylate monomer, triethylene glycol dimethacrylate, hydroxypropyl methacrylate, isobornyl methacrylate, isobornyl acrylate, hydroxyethyl methacrylate (HEMA), hema maleate, PEG-4 dimethacrylate, ethyl methacrylate, and dicyclopentadienylacrylate.

11. The composition, as recited in claim 1, wherein said visible light dryable nail coating is able to be wiped off from nail surfaces by a solvent.

12. The composition, as recited in claim 10, wherein said visible light dryable nail coating is able to be peeled off from nail surfaces.

13. The composition, as recited in claim 10, wherein said visible light dryable nail coating is able to be removed from nail surfaces by soaking into a solvent.

14. A method for applying visible light dryable nail coating, comprising the steps of:
(a) applying a visible light dryable nail coating on a nail surface; and
(b) curing said visible light dryable nail coating by a visible light source.

15. The method, as recited in claim 14, wherein in the step (a), a composition of said visible light dryable nail coating comprises a base acrylate copolymer, a photo-initiator, and a photo-initiator monomer.

16. The method, as recited in claim 15, wherein said base resin is cured by visible light, EB or heat

17. The method, as recited in claim 15, wherein said visible light photo-initiator has a wavelength between 400-450 nm.

18. The method, as recited in claim 15, wherein said visible light photo-initiator is Bis (η5-2,4-cylcopentadian-1-yl)-bis (2,6-di-fluoro-3-(1H-pyrrol-1-yl) phenyl) titanium.

19. The method, as recited in claim 15, wherein said visible light photo-initiator B is 4, 4′-Bis (diethylamino) benzophenone.

20. The method, as recited in claim 14, wherein said photo-initiator monomer is selected from the group consisting of one or more triethylene glycol dimethacrylate, hydroxypropyl methacrylate, isobornyl methacrylate, isobornyl acrylate, hydroxyethyl methacrylate (HEMA), hema maleate, and PEG-4 dimethacrylate, ethyl methacrylate, and dicyclopentadienylacrylate.

21. The method, as recited in claim 14, wherein in the step (b), said visible light dryable nail coating can be curved by any electromagnetic waves or particle beams which have wavelengths between 400-450 nm.

22. The method, as recited in claim 14, further comprising a step of:
(c) removing said visible light dryable nail coating from the nail surface in a solvent.

23. The method, as recited in claim 22, wherein said visible light dryable nail coating is removed from fingernails by peeling in just a single piece.

24. The method, as recited in claim 22, wherein said visible light dryable nail coating is wiped off by solvent remover.
